(19) United States
(12) Patent Application Publication
(10) Pub. No.: US 2008/0319476 A1
(43) Pub. Date: Dec. 25, 2008

(54) Hemostatic mineral compositions and uses thereof

(76) Inventors: Kevin R. Ward, Glen Allen, VA (US); Richard Brown, Billings, MT (US); Robert F. Diegelmann, Bon Air, VA (US); Gary L. Bowlin, Mechanicsville, VA (US)

Correspondence Address: VENABLE LLP, P.O. BOX 34385, WASHINGTON, DC, 20043-9998, US

(21) Appl. No.: 12/153,663

(22) Filed: May 22, 2008

Related U.S. Application Data
(60) Provisional application No. 61/006,978, filed on Feb. 8, 2008.
(60) Provisional application No. 60/939,384, filed on May 22, 2007.

(51) Int. Cl. A61B 17/03

(52) U.S. Cl. 606/213; 602/48

(57) ABSTRACT

The invention generally relates to compositions and methods for promoting hemostasis and sealing a wound by producing an adhesive cast. In particular, the invention provides compositions comprising clay minerals with specified particle sizes, which, when applied to a bleeding area, allow for a desired result in at least one of the following activities: stopping blood flow from a wound, forming a cohesive mass, sealing a wound, promoting coagulant activity, sorbing a body fluid, and adhering to tissue. For example, the desired result can be sealing the wound using an adhesive cast made of clay minerals mixed with blood or other wound fluids.

### BACKGROUND

1. Field

The invention generally relates to compositions and methods for promoting hemostasis and/or sealing a wound by producing an adhesive cast. In particular, the invention provides compositions comprising clay minerals with specified particle sizes, which, when applied to a bleeding area, allow for a desired result in at least one of the following activities: stopping blood flow from a wound, forming a cohesive mass, sealing a wound, promoting coagulant activity, sorbing a body fluid, and adhering to tissue. In some embodiments, the desired result is sealing the wound using an adhesive cast made of clay minerals mixed with blood or other wound fluids.

2. Background

Hemorrhagic events, from the minor to the life threatening, result from a wide variety of circumstances and occur in a wide variety of settings. The conditions which result in hemorrhage may be relatively predictable, such as those associated with medical procedures. Alternatively, hemorrhagic events may result from unpredictable circumstances, such as a breach of the skin or an internal organ in an accident. Such acute traumatic wounds occur in an almost infinite number of patterns and degrees, making the use of simple compression or application of a single type of bandage impractical if not impossible, especially in the most severe circumstances. For example, a traumatic wound to the groin cannot be readily controlled by simple direct pressure, by the use of a simple flat bandage, or by the use of a tourniquet.

Attempts have been made which partially address the treatment of hemostasis, and/or the need for flexibility in wound dressings:

1) Hemcon's Chitosan Bandage (see the website located at www.hemcon.com) is a gauze bandage impregnated with chitosan. Chitosan, a fiber derived from chitin in shellfish, is a nondigestible aminopolysaccharide. Chitosan is synthesized by removing acetyl groups from chitin, through a process called deacetylation. In models of life threatening hemorrhage (J Trauma 2005; 59:865-875 and J Trauma 2004; 56:974-983), the ability of the bandage to improve survival has been limited. In one study, involving isolated arterial injury, use of the bandage had a 100% failure rate. In a second study, involving combined arterial and venous hemorrhage at low blood pressures, the bandage resulted in a 28% mortality rate. It was noted in this study that there was a bandage-to-bandage variability in performance and ability of the bandage to adhere to the wound. This bandage is available in only one size and formulation.

2) The Fibrin Sealant Dressing (FSD) is the result of a collaborative effort between the U.S. Army and the American Red Cross. It is made from fibrin, thrombin, and factor XIII purified from human donated blood and plasma. It is thus a biologic which has a potential for disease transmission. The dressings come in bandage form and are fragile, tending to break apart if not carefully handled.

3) The Rapid Deployable Hemostat (RDH) is a bandage made by Marine Polymer Technologies and incorporates a derivative from marine algae to promote hemostasis. However, in a study by Alam and colleagues (Alam, et al: J Trauma 2003; 54:1077-1082), which explored the ability of many commercial products to stop severe bleeding and to increase survival, use of the RDH resulted in lower survival rates than a simple standard bandage.

4) U.S. Pat. No. 4,748,978 (to Kamp) discloses a therapeutic dressing that includes a flexible permeable support and a mixture of mineral and other components, including bentonite, kaolinite and illite or attapulgite, to treat burns and ulcers.

5) U.S. Pat. No. 4,822,349 (to Hursey et al.) describes a non-bandage material used to treat bleeding. The material is sold by Z-Medica as “Quick-Clot” (see the website located at www.z-medica.com) and is a granular form of zeolite, an aluminum silicate mineral. During use, it is poured into a wound. In addition to absorbing water from hemorrhaged blood and concentrating hemostatic factors in the blood at the site of injury, its mechanism of action appears to involve chemical cautery. An intense exothermic reaction is produced upon contact with liquid (e.g. blood), and is likely at least partially responsible for stoppage of blood flow by cauterization. While use of this material may be preferable to bleeding to death, the attendant burning of tissue at and near the wound (and possible burn injury of medial personnel who are administering the material) is a severe disadvantage. This side effect also reduces the ability of the material to be used for internal hemorrhage. Studies by Alam and colleagues (J Trauma 2004; 56:974-983) demonstrate that the ability of this product to stop hemorrhage is quickly lost when it is partially hydrated in attempts to reduce the exothermic reaction and the resulting temperature it produces in tissues. When the granules are placed in a bag similar to a tea bag to facilitate removal (“Quikclot ACS+”), its ability to stop bleeding is significantly limited.

6) A product made by TraumaDex (see the website located at www.traumadex.com) is a powder consisting of microporous beads which absorb water and which contain concentrated clotting factors. During use, the material is poured or squirted into the wound. However, when studied by Alam and colleagues (J Trauma 2003; 54:1077-1082) in a model of severe hemorrhagic shock, TraumaDex performed no better than a standard field dressing, thus offering no advantage and certainly more expense. Alam and colleagues studied this product again (J Trauma 2004; 56:974-983) and demonstrated its performance to be suboptimal compared to QuickClot and the Hemcon bandage. In this study, it performed only slightly better than a standard dressing.

A “one size fits all” approach to the treatment of hemorrhage clearly does not and cannot work, and the prior art has thus far failed to provide compositions and methods to treat hemorrhage that are safe, efficacious, highly adaptable, easy to use, inexpensive, and lacking in serious side effects.

### SUMMARY

Embodiments of the present invention are directed to mixtures of clay particles of different specified particle sizes that have been selected to allow for a desired result in at least one of the following activities: stopping blood flow from a wound, forming a cohesive mass, sealing a wound, promoting coagulant activity, sorbing a body fluid, and adhering to tissue.

These compositions can be sterilized and packaged to form compositions for use in stopping blood from a wound, for example, hemorrhaging wound. The compositions described herein can be loose powders, loose granules, or a powder and/or granule mixture that has been combined with, adhered to, and/or enclosed by or suspended within a substrate. Other embodiments relate to the design, production, and use of the compositions described herein.

### BRIEF DESCRIPTION OF THE DRAWINGS

FIG. 1 illustrates an example particle size distribution, on a cumulative percent passing basis, of some embodiments of the invention described herein.

FIG. 2 illustrates an example particle size distribution, on a incremental percent retained basis, of some embodiments of the invention described herein.

FIG. 3 illustrates the exothermic activity of Quikclot ACS+ and an example embodiment of the invention (a version of the WoundStat™ (WS) product).

FIG. 4 illustrates the percent survival and survival times between treatment groups. Seven of seven animals treated with WS survived the two hours of observation while no animal treated with QuikClot® Granules (QCG) survived. The difference between the WS and QCG in survival and survival times was significant with p=0.0005 and p=0.001 respectively.

FIG. 5 illustrates the Mean Arterial Pressure (MAP) of test animals in research testing over a two hour test period. There was no significant difference between the groups at baseline, immediate post hemorrhage, and immediate post application at times. At 15 minutes post hemorrhage and beyond, the difference between WS and QCG had a significance of p<0.001 until approximately 70 minutes when the only surviving QCG animal temporarily increased its MAP prior to sudden cardiovascular collapse.

FIG. 6 illustrates the peak post-application wound temperatures. At post application, the wound temperature was significantly different between WS (33.4±4.7° C.) and QCG (63.6±17.4° C.).

### DETAILED DESCRIPTION

The contents of WO/2006088912 are incorporated herein in their entirety. All other articles, patents, publications, or webpages mentioned herein are also incorporated in their entirety.

Embodiments of the invention described herein provide compositions comprising clays, clay minerals, and/or related materials having specific particle sizes, and methods for their use in treating and controlling hemorrhage, i.e., in promoting hemostasis. The inventors have discovered that the ability of a clay composition to effectively clot blood and/or control a hemorrhaging wound is due, at least in part, to the particle size of the clay or mixture of clays used in the composition.

It is believed that these compositions can act in a variety of ways to promote hemostasis in a bleeding wound. For example, when administered to a bleeding or high pressure hemorrhaging wound these compositions form a tight seal that closes the wound and also applies pressure to the wound. This pressure generation can be further enhanced by applying pressure to the composition after it has been packed or placed into the wound.

The terms “hemorrhage” or “acute hemorrhage” mean the loss of blood from one or more anatomical sites of a patient that, if left untreated, would jeopardize the health of the patient. Hemorrhage typically results from rupture of one or more blood vessels, which may occur accidentally (e.g. as in accidental wounds) or purposefully (e.g. during surgical procedures). A hemorrhaging wound can involve blood flow leaving the wound at a high pressure making the hemorrhaging wound difficult to seal.

The active control of hemorrhage is referred to as “hemostasis.” In some embodiments, “hemostasis” refers to the cessation of bleeding from a wound. The promotion of hemostasis involves, for example: slowing or stanching the flow of blood (e.g., through direct pressure and/or mechanical means such as a tourniquet or cast); and enhancing, facilitating or causing the blood to clot, particularly at the site of a wound.

The term “clay,” as used herein, refers to natural or synthetic material, composed primarily of fine grained minerals, which is generally plastic at appropriate water contents and will harden when dried or fired. Those skilled in the relevant arts will recognize that, while clay usually contains members of the phyllosilicate mineral group, it may contain other materials that impart plasticity and harden when dried or fired as well as associated mineral phases that do not impart plasticity, and organic matter. The term “clay minerals” means naturally occurring or synthetic phyllosilicate minerals as well as minerals that impart plasticity to clay and which harden upon drying and firing.

In some embodiments, the clay is selected from, but not limited to, the following: bentonite, montmorillonite, beidelite, nontronite, saponite, hectorite, illite, illite-smectite mixed layer clay, sepiolite, attapulgite (palygorskite), kaolin or kaolinite or mixtures thereof.

In some embodiments of the invention, the materials are naturally occurring clays referred to as bentonites. Bentonite is a clay consisting predominately of smectite minerals, especially montmorillonite. Bentonite may also refer to sodium bentonite, western bentonite, Wyoming bentonite, sodium montmorillonite, calcium bentonite, southern bentonite, calcium montmorillonite, taylorite, fuller's earth, and a variety of commercial trade names. There are three major types of commercial bentonite: 1) natural calcium bentonite; 2) natural sodium bentonite; and 3) sodium activated calcium bentonite. The term “bentonite” as used herein is intended to encompass all synonyms and all types of bentonite, unless otherwise specified.

In some embodiments of the invention, the clay that is used comprises kaolin. One known use of kaolin is in the common coagulation test called the “activated partial thromboplastin time” which is a measure of the activity of the intrinsic clotting system. The activator for this test is kaolin.

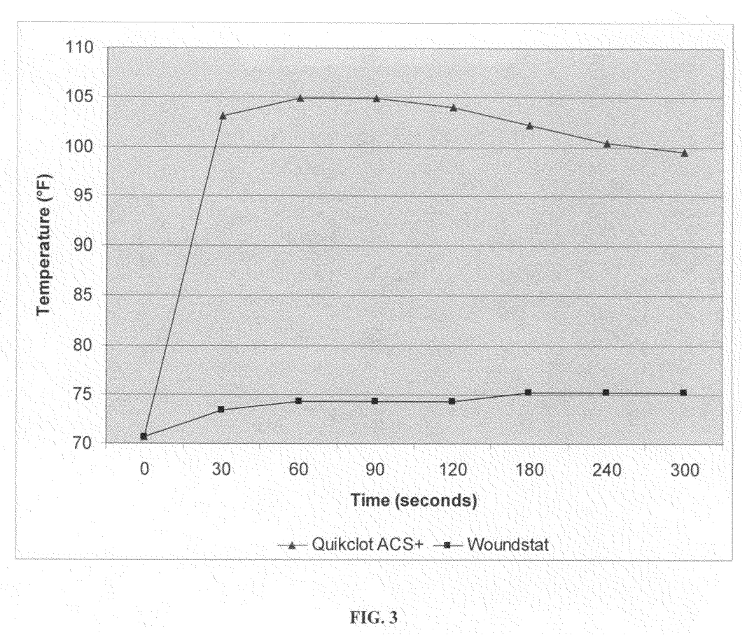

The clays used in the present invention do not exhibit significant exothermic activity when placed in an aqueous environment, such as a bleeding wound. As seen in Table 1 and FIG. 3, the test mixture of one embodiment of the present invention produces much less heat in an aqueous environment than a zeolite based product, for example, QUIKCLOT® ACS+. Clays according to the invention generally do not produce a temperature rise significantly above body temperature when applied to a wound.

TABLE 1

| | |
|---|---|
| Quikclot ACS+ | WS Test Mixture |

| Time | Temp | Temp | Temp | Temp |
|---|---|---|---|---|
| (sec) | (° C.) | (° F.) | (° C.) | (° F.) |
| 0 | 21.5 | 70.7 | 21.5 | 70.7 |
| 30 | 39.5 | 103.1 | 23 | 73.4 |
| 60 | 40.5 | 104.9 | 23.5 | 74.3 |
| 90 | 40.5 | 104.9 | 23.5 | 74.3 |
| 120 | 40 | 104 | 23.5 | 74.3 |
| 180 | 39 | 102.2 | 24 | 75.2 |
| 240 | 38 | 100.4 | 24 | 75.2 |
| 300 | 37.5 | 99.5 | 24 | 75.2 |

The particles of the present invention have desirable sorptive properties. The terms “sorb” and “sorptive” refer to the ability of a particle to take up a liquid either by adsorption, by absorption, or by a combination of both. For example, the particles of the present invention can be used to sorb blood. The particles of some embodiments of the present invention can sorb blood in amounts up to about ten times their dry weight. In some embodiments, the particles of the present invention can absorb blood, adsorb blood, or adsorb and absorb blood when applied to a wound.

Some selected clay minerals have been found to have a remarkable and unexpected ability to cause blood to clot. Even heparinized blood will clot in their presence. Without being bound by theory, it is noted that the distribution of cations and anions in this type of material may cause favorable hemostasis, since cationic species are known to cause red cell aggregation and hence clotting, perhaps through a cation exchange mechanism. The negative charge of the clay may also activate the intrinsic clotting system.

The clay compositions utilized in the present invention may include one or more clay minerals, i.e., a mixture of clays may be utilized. Those of skill in the art will recognize that such mixtures may occur naturally, in that deposits of clays may or may not be composed of only one type of clay mineral. Alternatively, the mixtures may be formed purposefully during production of the compositions.

In addition to recognizing the ability of clay to clot blood, the inventors have discovered that mixing specific amounts of larger and smaller particles of clay changes the ability of a clay composition to control a hemorrhaging wound. The quantity and size of the clay particles selected for the hemostatic compositions can influence numerous desirable properties for treating a hemorrhaging wound including, but not limited to: sealing of the wound, procoagulant activity (e.g., promoting coagulant activity), the adsorption of fluid (e.g., blood), adherence of the composition to tissue, flexibility of the composition, permeability of the composition, cohesion of the particles in the composition to one another, the ability of the composition to apply pressure to the bleeding wound, and other desirable characteristics. The selection of particle size(s) can also be changed to impact the ability to reuse the composition and/or remold it after is has been applied to a wound. The relative importance of each property can vary based on the type of wound being treated and/or the hemostatic application for which the clay composition will be used. Accordingly, embodiments of the present invention include mixtures of larger and smaller clay particles in a measured amount for use in controlling blood loss, e.g., treating a hemorrhaging wound.

While there is no specific boundary between large and small clay particles, e.g., bentonite, generally particles larger than about ¼″ (which are called bentonite chips or gravel and are often used for well sealing) can be considered as large and particles of ground bentonite, on the order of 100 mesh or smaller, can be considered as small. The particle size ranges combined in the present invention relate primarily to particles having a size between these two extremes. Although this example describes bentonite, one of skill in the art will appreciate that other clays may be used as part of the present invention.

As one of skill in the art will appreciate, the particle size of a clay can be determined using standardized sieving techniques. For example, US standard or ASTM sieve sizes can be used to describe the size of particles. These sieve measurements can be converted into micrometer measurements, if desired, using readily available conversion tables, for example, at www.humboldtmfg.com/sieves.php?sievenum=1 or www.reade.com/en/Reference-% 10-Educational/Particle-Measurement/International-Sieve-Chart-% 10-Micropowder-Grit-Chart.html.

One of skill in the art will appreciate that the sieve sizes used herein relate to the practice of mechanical sieving, either during production or for measuring the result of production. For example, some embodiments of the invention use the API (American Petroleum Institute) 13B testing protocol, which is, essentially, the same as ASTM method D6913-04. Specifying a sieve size, by default, also specifies a size in micrometers, which can also be determined by standard light diffraction techniques employed by a variety of commercially available particle sizing test equipment.

The compositions of the present invention, in some embodiments, begin by selecting the desired particle sizes of the desired clay for use in the composition. These particles are also referred to as “granules” and the two terms are intended to be synonyms. The clay is extracted from the earth, dried to have a moisture content of between about 1% to about 24%, or about 5% to 15% or more preferably about 6% to 9%, and then passed through one or more sieves to select particles of a particular size. The mixture that passes through any particular sieve has a particle size less (or no greater) than that of the opening in that sieve. The desired particle size distribution of the compositions of the present invention may be directly achieved by selective drying, crushing, and screening of the clay.

Particles of differing particle sizes, or different particle size ranges, can also be blended together in varying amounts or ratios, e.g., via back blending, to produce the compositions useful for treating a hemorrhaging wound. For example, larger particles can be blended with smaller ones in varying ratios, amounts, or percentages. As one of skill in the art will appreciate, particle size ranges can also be produced by blending two or more granular clay products having different particle size distributions to achieve the desirable particle size distribution or the desired particle size.

The compositions described herein can include, but are not limited to, those compositions containing:
1. Mixtures of clay particles where at least about 90% of the particles having a particle size of less than 4 mesh and about 5% of the particles having a particle size of less than 100 mesh;
2. Mixtures of clay particles where at least about 95% of the particles having a particle size of less than 12 mesh and about 10% of the particles having a particle size of less than 100 mesh; and/or
3. Mixtures of clay particles where at least about 100% of the particles having a particle size of less than 12 mesh, about 35% to about 50% of the particles having a particle size of less than 40 mesh, and about 15% of the particles having a particle size of less than 100 mesh.
4. Mixtures of clay particles of a size from about 12 mesh to about 200 mesh where the particles from 12 mesh to 40 mesh represent from 40% to 80% of the total on a weight basis (e.g., Big Horn #34 in the Example which is about 50 to 60%+40 mesh).
5. Mixtures of clay particles with particles as large 4 mesh with a gradation of particle sizes down to about 200 mesh.

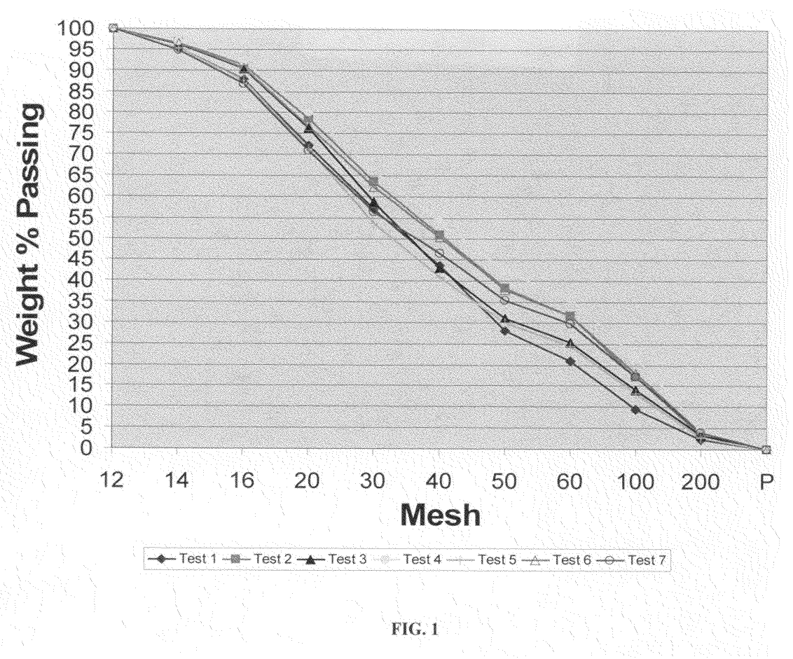

As illustrated in FIG. 1, another preferred particle size mixture forms a roughly even distribution throughout the range of particle sizes with about one third of the particles being between 12 mesh (1,700 μm) and 22 mesh (˜794 μm), one third between 22 mesh and 55 mesh (˜275 μm) and one third being smaller than 55 mesh. This data is also presented in the “Cumulative % Passing Screen” portion of Table 2 below.

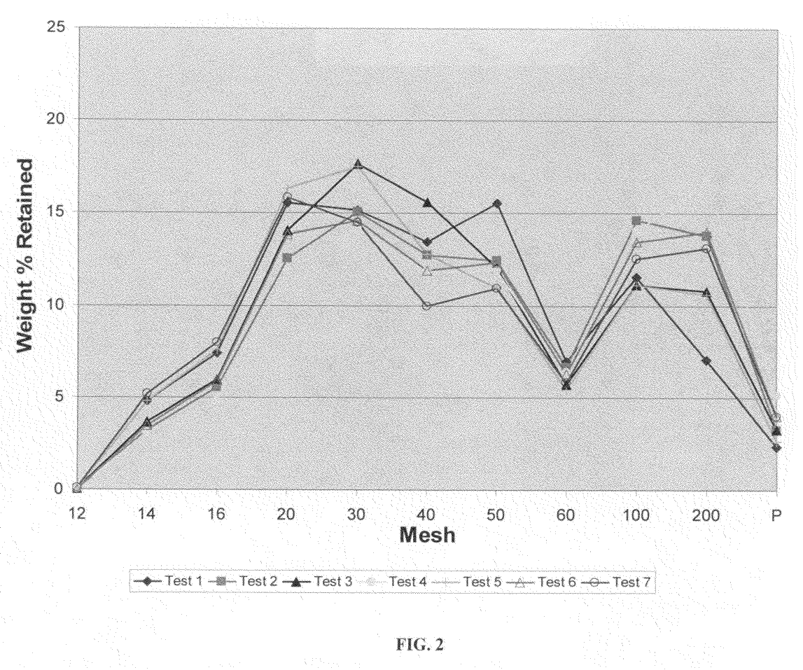

FIG. 2 illustrates some of the variability in the particle size distribution of some of the embodiments of the invention. This data is also presented in the “% On Screen” portion of Table 2 below.

Some embodiments of the present invention can include at least as part of the composition a sterilized form of the following blends or mixtures of Big Horn Bentonite™ (available from Wyo-Ben, Inc., Billings Mont.): (1) #8=particles in the range between 4 mesh (4,750 μm) and +12 mesh (1,700 μm) (−4+12 mesh); (2) #16=particles in the range between 8 mesh (2,360 μm) and 32 mesh (500 μm) (−8+32 mesh); (3) #30=particles between 12 mesh (1,700 μm) and 32 mesh (500 μm) (−12+32 mesh); (4) #40=particles less than 32 mesh (500 μm) (−32 mesh); (5) #34=a blend of approximately 55% #30 and 45% #40; (6) #200=a fine ground product (powder) where approximately 80% of the particles are less than 200 mesh (75 μm) (80%-200 mesh). Some embodiments can also include low adsorption bentonite, which is sodium bentonite that, because of its unique crystal structure and chemistry, has a significantly lower capacity to sorb water and swell, than other sodium bentonite and which is on the order of or slightly higher than the capacity of a typical calcium bentonite. In some embodiments, sterilized FS-34 can be used as part of the present invention.

As one of skill in the art will appreciate, other embodiments of the invention, even if not listed above, can be determined based upon the method disclosed herein of providing a dry clay particle matrix having a sufficient number of interconnected, interparticle voids with a sufficient void volume to provide sufficient permeability within the dry mass of clay particles to allow rapid blood penetration through the clay mass to ensure rapid and substantially complete wetting and activation of the clay particles. The size of the individual voids and overall void volume of the clay mass should be controlled to ensure that the mass is substantially self void filling when wetted with blood in a wound and retains sufficient particle to particle cohesion to provide good structural integrity to the wetted mass. The mass should also remain somewhat pliable and adhere well to the wound tissue to enable it to stay in place and resist normal blood pressures to prevent bleeding.

After the initial sieving and/or blending has been completed, testing can be done to identify additional details about the size distribution of the particles in the composition and confirm that the proper particle sizes have been selected. An example of the results of such testing is presented in Table 2.

TABLE 2
Testing of sample embodiments
Particle Size Comparison

| Sieve | Test | Test | Test | Test | Test | Test | Test |
|---|---|---|---|---|---|---|---|
| Size | 1 | 2 | 3 | 4 | 5 | 6 | 7 |
| | % on Screen | | | | | | |
| 12 | 0.04 | 0.06 | 0.04 | 0.05 | 0.05 | 0.02 | 0.09 |
| 14 | 4.78 | 3.23 | 3.69 | 3.1 | 4.81 | 3.49 | 5.18 |
| 16 | 7.43 | 5.5 | 5.97 | 4.78 | 7.61 | 5.87 | 7.97 |
| 20 | 15.57 | 12.53 | 14.04 | 11.89 | 16.33 | 13.84 | 15.85 |
| 30 | 15.17 | 15.08 | 17.62 | 14.13 | 17.49 | 14.58 | 14.49 |
| 40 | 13.47 | 12.72 | 15.62 | 11.88 | 12.81 | 11.94 | 9.96 |
| 50 | 15.55 | 12.46 | 12.05 | 11.9 | 11 | 12.37 | 10.93 |
| 60 | 6.96 | 6.7 | 5.7 | 6.39 | 5.44 | 6.31 | 5.83 |
| 100 | 11.57 | 14.62 | 11.14 | 14.05 | 11.15 | 13.49 | 12.53 |
| 200 | 7.07 | 13.81 | 10.81 | 16.7 | 10.6 | 14.01 | 13.14 |
| P | 2.37 | 3.29 | 3.29 | 5.13 | 2.68 | 4.04 | 4 |
| | Cumulative % Passing Screen | | | | | | |
| 12 | 99.96 | 99.94 | 99.96 | 99.95 | 99.95 | 99.98 | 99.91 |
| 14 | 95.18 | 96.71 | 96.27 | 96.85 | 95.14 | 96.49 | 94.73 |
| 16 | 87.75 | 91.21 | 90.3 | 92.07 | 87.53 | 90.62 | 86.76 |
| 20 | 72.18 | 78.68 | 76.26 | 80.18 | 71.2 | 76.78 | 70.91 |
| 30 | 57.01 | 63.6 | 58.64 | 66.05 | 53.71 | 62.2 | 56.42 |
| 40 | 43.54 | 50.88 | 43.02 | 54.17 | 40.9 | 50.26 | 46.46 |
| 50 | 27.99 | 38.42 | 30.97 | 42.27 | 29.9 | 37.89 | 35.53 |
| 60 | 21.03 | 31.72 | 25.27 | 35.88 | 24.46 | 31.58 | 29.7 |
| 100 | 9.46 | 17.1 | 14.13 | 21.83 | 13.31 | 18.09 | 17.17 |
| 200 | 2.39 | 3.29 | 3.32 | 5.13 | 2.71 | 4.08 | 4.03 |
| P | 0.02 | 0 | 0.03 | 0 | 0.03 | 0.04 | 0.03 |

In some embodiments, the compositions of the present invention are sterilized or sterile. As used herein, the terms “sterilized” and “sterile” refer to compositions free of microbes including bacteria, fungi, and/or viruses or a composition that has passed a standard sterility test. For example, the compositions can be sterilized using radiation, heat, or treatment with various gaseous agents known to one of skill in the art without disrupting the desirable characteristics of the compositions, e.g., the particle size and/or moisture content.

One exemplary process for sterilizing the compositions in bulk can involve:

(1) Pallets of filled and sealed pouches (or other container) containing the composition arrive in “shippers” (cartons) each containing 64 pouches and are unloaded.

(2) The shippers can be placed into “cells” which are moved into the radiation chamber for Gamma ray radiation.

(3) Any dose of radiation that is sufficient to sterilize the product may be used. For example, the radiation dose can be between about 35 kGy and 100 kGy. In some instances, more than one run of radiation is necessary. For example, two or more runs of radiation with the cells can be used.

(4) After the pouches containing the composition have been sterilized the shippers are unloaded from the cell, repalletized and shipped to consumers.

Some embodiments of the present invention use formulations of particles with specific particle sizes for the direct application of the particles to a wound. These particles can be in the form of a loose powder or mixture of granules. These formulations can be applied directly to a bleeding wound. This application of a loose powder or a mixture of granules can be used to fill the cavity of the wound, seal the ruptured blood vessel, and/or form an adherent seal within the wound or on top of the wound.

It has been discovered that such compositions can effectively seal a wound and stop bleeding even without direct contact with the ruptured blood vessel. For example, gauze was placed in the base of a wound to prevent direct contact of the clay particles with the blood vessel. Application of the clay particles of the present invention to the wound on top of the gauze sealed the wound and achieved hemostasis.

The compositions of some embodiments of the present invention were also able to achieve hemostasis in wound where the blood vessel was ruptured on the posterior side (away from the application of the clay particles) despite the clay particles not coming in direct contact with the hole in the blood vessel.

The compositions of the present invention can, in some embodiments, be affixed, enmeshed, intertwined, coated onto, or otherwise adhered to a substrate. The substrate may be composed of any suitable material, either natural or man-made and organic or inorganic, e.g., cotton, wool, linen, rayon, nylon, polyester, polyethylene, mineral wool or metal fibers, or blends of these materials, and may be in any suitable form, e.g., formed meshes, grids or matrixes, woven fabrics or nonwoven fabrics, as well as mixtures of these forms, that is suitable for, and may facilitate the use of, the compositions of the present invention. It should be understood that the examples given should not be interpreted to limit in any way the range of substrates that are provided herein.

The composition may consist entirely of clay or a variety of other compounds or materials may be added to the clay, examples of which include antimicrobial agents (e.g. antibiotic, antifungal, and/or antiviral), electrostatic agents (e.g. dendrimers in which the charge density is varied or similar compounds), preservatives, various carriers which modulate viscosity, various colorants, and various medicaments which promote wound healing. Other appropriate hemostatic or absorptive agents may also be added. These include but are not limited to chitosan and its derivatives, fibrinogen and its derivatives (represented herein as fibrin(ogen), e.g. fibrin, which is a cleavage product of fibrinogen), super-absorbent polymers of many types, cellulose of many types, alkaline earth cations such as iron, calcium, and sodium, metallic cations such as silver, or various anions, other ion exchange resins, and other synthetic or natural absorbent entities such as super-absorbent polymers with and without ionic or charge properties. In some embodiments of the invention, exchangeable cations of one type on the clay may be substituted with cations of another type (e.g. silver cations)

In addition, the clay mineral may have added to it vasoactive or other agents which promote vasoconstriction and hemostasis. Such agents might include catecholamines or vasoactive peptides or agents such as chitosan, thrombin, etc. This may be especially helpful in its dry form so that when blood is absorbed, the additive agents become activated and are leached into the tissues to exert their effects. These agents may be coated onto the particles of the clays via processes like spray drying. In addition, antibiotics and other agents which prevent infection (any bactericidal or bacteriostatic agent or compound) and anesthetics/analgesics may be added to enhance healing by preventing infection and reducing pain. In some embodiments, agents such as copper or silver, which have antibacterial properties, are included within the compositions.

In addition, fluorescent agents, radioisotopes, or other components could be added to help during surgical removal of some forms of the mineral to ensure minimal retention of the mineral after definitive control of hemorrhage is obtained. These could be viewed during application of light for example from a Wood's lamp. In short, any suitable material may be added, so long as the clay composition is still able to cause blood clotting and/or promote hemostasis.

Some embodiments of the invention include unit packages of a measured amount of the mixture of clay particles. The unit package can be, but is not limited to, a pouch, sachet, sack, bag, box, can, bottle, tube, or other equivalent container capable of holding a measured amount of clay. The unit package can be a single use package or part of a multi-pack.

The measured amount of the clay particles varies depending on the hemostatic application the composition will be used for but can be between about 0.01 grams to about 250 or more grams. For example, embodiments can use a measured amount of about 0.01, 0.1, 1.0, 5, 10, 15, 20, 25, 50, 100, 200, or 250, or more, or less, grams. The unit package with the measured amount will generally be less than 1 kg or 500 g in weight.

Also, the measured amount of the clay can be from about 1 ounce to about 20 ounces. For example, the measured amount can be about 2, 3, 4, 5, 6, 7, 8, 9, or 10 ounces.

As discussed above, the unit package can hold a sterilized composition and is designed to preserve the sterile condition of its contents until use. The unit packages can also be designed and/or packaged in a manner that will prevent the particles of clay from being broken down, degraded, contaminated, dried, or hydrated during shipping, storage, or during or prior to use of the composition.

In some embodiments, it is necessary to ensure that the homogenous mixture of clay particle sizes found to be useful (and produced by the clay producer) is not altered by differential segregation during packaging to produce a multiplicity of heterogenous mixtures in the packages. Re-blending (re-homogenization) of the product prior to or during packaging can be necessary if undesirable segregation is found to occur.

Further, in some embodiments, the range of particle size produced by the clay producer should not be altered. Clays are inherently soft materials and subject to particle degradation during handling. This can be controlled during shipping and packaging to prevent the range of particle sizes produced from changing to finer sizes which would not be advantageous for the intended use. Additionally, it can be useful to control the moisture content of the produced clay product to ensure that it does not sorb moisture from the atmosphere or from contact with liquid water causing the clay granules to agglomerate into larger particles.

The production and particle selection methods described herein allow a predetermined, consistent mixture of clay particles to be produced. These methods provide an improved product that has consistent, predictable, and reproducible results when used in the field.

The compositions, formulations, and unit packages described herein are useful in methods of treating a hemorrhaging wound, promoting hemostasis in a wound, and/or other conditions related to the loss of blood or other fluids (e.g., lymph). These methods can be used on any animal, mammal, or in particular human, in need of treatment.

The compositions and formulations of the present invention may be administered to a site of bleeding by any of a variety of means that are well known to those of skill in the art. Examples include, but are not limited to, internally, directly to a wound, (e.g. by pouring or shaking powdered or granulated forms of the material directly into or onto a site of hemorrhage, followed by kneading if necessary), by placing a material such as a bandage that contains or is impregnated with the material into or onto a wound, or otherwise coating the wound with the material.

Many applications of the present invention are based on the known problems of getting the surfaces of bandages to conform to all surfaces of a bleeding wound. The use of granules and/or powders allow the preparations of the invention to cover all surfaces no matter how irregular they are. For example, a traumatic wound to the groin is very difficult to control by simple direct pressure or by the use of a simple flat bandage. However, treatment can be carried out by using clay in the form of, for example, a powder or granule preparation that can be poured into the wound, followed by application of pressure if needed. One advantage of the preparations of the present invention is their ability to be applied to irregularly shaped wounds, and for sealing wound tracks, i.e. the path of an injurious agent such as a bullet, knife blade, etc.

Compositions comprising clay may be utilized to control bleeding in a large variety of settings, which include but are not limited to:

a) External bleeding from wounds (acute and chronic) through the use of powder, granules, or the coating of bandages with these preparations.

b) Gastrointestinal bleeding through the use of granules or powder.

c) Epistaxis through the use of an aerosolized powder, patches, or coated tampon.

d) Control of internal solid organ (e.g., liver or spleen) or boney injury through the use of powder; granules; or bandages having powder or granules enmeshed in the bandage, intertwined with the bandage, coated onto the bandage, or otherwise adhered to the bandage.

e) Promotion of hemostasis, fluid absorption and inhibition of proteolytic enzymes to promote healing of all types of acute and/or chronic wounds including the control of pain from such wounds.

The compositions, formulations, and unit packages described herein are also useful in methods of forming a cast to cover, close, seal, or otherwise stop the bleeding from a wound. These methods involve applying a sterile composition described herein in a quantity sufficient to form a cast over the wound. The cast is formed from one or more clay minerals and blood from said hemorrhaging wound. The cast can be pliable or rigid, as clinical conditions dictate. As described in the examples below, the pliability of the cast formed can be controlled by the selection of clay particles having certain particle sizes and including them in the composition used to form the cast. These casts are particularly advantageous for battlefield conditions because they can be administered to a wounded person quickly, form a cast rapidly, and have sufficient pliability to remain over the wound until the wounded person can be taken to a hospital for additional care.

The formation of the cast can be done, in some embodiments, by applying the compositions described herein directly to the wound. For example, a granular product can be poured directly into or onto the wound, kneaded to more rapidly or completely incorporate the blood or other body fluids into the granular clay if required, and allowed to seal the wound for the required amount of time. Once the clay has become sufficiently wetted and has developed sufficient cohesion between clay particles and adhesion to the wound tissue a durable, pliable cast is formed and the blood flow will be stopped.

In some embodiments, the pliable cast can consist essentially of blood mixed with the clay but also will include smaller amounts of other fluids absorbed from the wound (e.g., lymph).

This stoppage of blood flow in a wound using the compositions described herein can be attributed, at least in part, to the formation of a tight, adhesive seal between the tissue surrounding the wound and the edges of the cast, the formation of a tight, adhesive seal between the ruptured blood vessel and the composition within the wound, and to the pressure imparted to the wound by the presence of the cast itself. The adhesive and sealing qualities of the cast, as well as its adsorptive and absorptive characteristics, can be controlled by the selection of specific particle sizes for inclusion in the composition. In some embodiments, the compositions described herein can stop bleeding and/or promote hemostasis in under 2 minutes or under 1 minute after being applied to a hemorrhaging wound.

The formation of a cast in the wound can generate pressure in the wound either individually or in combination with external pressure applied to the composition after it has been packed into the wound. Such wound pressures applied by the cast have been observed to exceed 100 mmHg in test animals with a ruptured femoral artery. This pressure is above the systolic pressure of the animal indicating that that pressure on the artery exceeds the intraluminal hydrostatic pressure thereby resulting in the stoppage of blood flow through the vessel by the external pressure exerted on it by the molded clay composition in the wound. See Acheson et al., Journal of Trauma 2005:59, 865-74 for a description of the experimental methods.

In some embodiments, the pressure exerted by the composition once packed into the wound substantially remains even after manual pressure being applied to the wound (e.g., a medic pressing gauze on the wound to stop bleeding) is removed. This application of pressure from the composition after being packed into the wound can stop bleeding even without clotting of the blood, making these compositions desirable to persons who cannot effectively clot blood (e.g, coagulopathic patients) or are taking blood thinning medications. The compositions can be used on patients with congenital or acquired coagulopathy, which refers to a defect in the body's mechanism for blood clotting. An example of a congenital coagulopathy is hemophilia. An example of an acquired coagulopathy includes persons who take warfarin and cannot clot blood. As one of skill in the art will appreciate, these examples of coagulopathy are not limiting.

The compositions of some embodiments of the present invention were able to achieve hemostasis in a wound having diluted blood with hemoglobin levels of less than 2 g/dl, indicating severe hemo-dilution and anemia. Yet, the application of clay particles of the present invention to the wound containing diluted blood resulted in hemostasis in less than two minutes.

The compositions of some embodiments of the present invention were also able to stop blood loss in a hemorrhaging wound in the presence of saline solution and very little blood. Bleeding was produced from the femoral artery of a pig and then the wound clamped closed to stop the bleeding. The blood within the wound was suctioned out and replaced with saline solution. Clay particles of the present invention were packed into the wound. The vascular clamp was then released to allow blood flow from the ruptured blood vessel. Hemostasis was achieved in the absence of any significant amount of blood in the wound.

In some embodiments, the clay composition used for generating pressure in the wound is in the form of granules, a bandage impregnated or otherwise coated with clay as described herein, a perforated pouch or mesh bag containing clay, or other form described herein. Such bags or pouches may be made of a dissolvable material such as pullulan, dextran, gelatin, cellulose-derivatives, hydrocolloids, polysaccharides, or mixtures thereof. Thus, the clay particle mixture may be either loose or fixed.

In some embodiments, the clay composition used for generating pressure in the wound is in the form of particles of clay contained within a sealed, un-perforated pouch or bag composed of a water soluble material. The term “water soluble” as used herein includes compositions that are dissolvable or otherwise dispersible in water.

The water soluble material can be a water soluble plastic. Suitable water soluble plastics include, but are not limited to, polyvinyl alcohol, ethylcellulose, hydroxypropyl methylcellulose or polyethylene oxide, or mixtures thereof. In some embodiments, the water soluble or dissolvable material can be a film.

In some embodiments, the water soluble or dissolvable substrates containing clay can be applied to a wound and the water soluble or dissolvable material will dissolve in the wound fluids including blood. The water soluble or dissolvable substrate can be formed into a container of suitable shape to contain the sterile composition and allow it to be conveyed to a wound as an intact mass.

Such substrates can be packaged within an exterior container as described herein (e.g., a foil package) to preserve the structure and sterility of the composition until use. Those compositions and packages that can be used to treat a wound or in another medical use are considered to be “suitable for medical use.”

Additives may optionally be mixed with the clay particles in the composition to enhance the composition's ability to generate pressure by increasing inter-clay particle adherence and/or adherence of the clay particles at the site of the bleeding. Such additives include, but are not limited to, polyacrylamides, polysaccharides, polyacrylates, muco-adhesive compounds, and mixtures thereof.

The embodiments that generate pressure in the wound can be used in a wide variety of medical situations. For example, to promote hemostasis in a hemorrhaging wound. These compositions are useful in rainy or high moisture battlefield conditions because they can effectively seal the wound despite an elevated water content in and around the wound area. Such elevated water content during tactical situations can impair the ability of pro-coagulant devices and compositions by washing away the active ingredients or diluting their effects in the wound.

In addition to the description above, the following non-limiting example further illustrates the invention described herein.

### EXAMPLE 1

As outlined below, testing was conducted on various different particle size granular bentonite compositions to create a composition that allowed for rapid, uniform, and complete blood penetration into, and wetting of, a mass of product placed in a wound. These compositions were developed to have sufficient cohesion between the wetted clay particles to form a structurally competent cast of clay with sufficient adhesion and sealing to allow the clay cast to adhere to the tissue of the wound and remain adhered until removed, e.g., by a medical professional.

The compositions were also tested to determine if they would fracture in a brittle fashion when placed in a wound and wetted or would remain pliable so that the mass in the wound could move with the wound tissue. The compositions were also tested to determine if they would require finger kneading in the wound to encourage complete wetting with blood.

Sample Preparation:

For these tests, the geometry of the wounds that are traditionally made to expose the femoral artery of pigs, as part of the standard model of a hemorrhaging wound, were duplicated. These wound openings (along the crease between the abdomen and the leg to expose the femoral artery) were roughly 4 to 6 inches in length, 1 to 2 inches in depth at the deepest point, and 3 to 4 inches wide at the widest point. When filled with blood the wound had an approximate volume of 156 cc. The wound geometry was approximated using a standard, 5 gallon plastic bucket tilted at a 45 degree angle. The crease formed between the bottom and side of the bucket, when tilted at this angle, provided approximately the same geometry as that of the wounds in the test animals.

Each test was conducted by pouring 156 cc of tap water into the crease of the tilted bucket to simulate a blood filled wound. 156 gm of each of the various granular bentonite test samples was then rapidly poured into the water in the bucket crease, with a 4″ wide, flat-bottomed, plastic feed scoop, using a side-to-side shaking motion, to help to ensure an approximately even distribution of the bentonite across the full area occupied by the water.

A stop watch was started immediately upon pouring the bentonite into the water. The time required for all the water to be sorbed by the bentonite, up to a limit of 60 seconds, was noted for each sample. At the end of 60 seconds any remaining, un-sorbed water was carefully poured from the bucket and its volume measured. The now-swollen mass (cast) of hydrated bentonite was carefully cut away from the sidewall and bottom of the bucket, using a metal spatula, so as to maintain the mass in one piece having the original form from the bucket crease, and to avoid losing any of the clay from the mass. The bentonite mass was then removed from the bucket, inverted over a collecting dish, and gently shaken to remove any un-wetted clay. The un-wetted clay was then weighed and the weight recorded. The remaining bentonite mass was then set aside for further investigation.

Method Variations:

For some tests the bentonite was manually kneaded for 15 seconds, immediately after placing it in the water, to ensure complete hydration after which, the flat of the palm of the hand was placed on the bentonite to apply some pressure to the mass for the remainder of the 60 second wetting period (Sample Preparation (SP) Type I). For other tests no kneading or pressure were used and the bentonite was merely allowed to freely sorb the water without disturbance (SP Type 2).

Wetted bentonite masses were tested in the following ways:

Test Type 1

Wetted masses were held between the thumb and forefinger while pressure was applied between the fingers. This was tried at several points along the length of each mass and the pliability (plasticity) of each mass was subjectively rated on a 1 to 4 scale with 1 being the most pliable and 4 being the least pliable.

Test Type 2

Wetted masses were sliced perpendicularly to their long axis, using a wire-type cheese slicer, to produce individual sections having a thickness of 1″. These sections were then trimmed with a sharp knife to a width of ¾″. Each section produced in this fashion was then placed on a sample support located on the test pad of a Chatillion Model DPP-5 manual Mechanical Force Tester equipped with a Chatillon Model AC-384-1 Mechanical Force Gauge. The sample support consisted of a “U” shaped piece of 1/16th″ thick steel strapping ¾″ tall by 2″ long with a 1″ gap between the sides of the “U”.

The mass sample was placed perpendicularly across the long direction of the support at the mid point of the length of the sample and the support. The sample and sample support were placed on the test pad of the Force Tester so that the gap of the test support was directly under the 1″ long×¼″ wide, rectangular pressure foot of the force tester. The test pad was then raised, by manually depressing the actuating level on the Force Tester, until the pressure foot of the Force gauge just touched the test sample. The Force Tester test pad was then further raised by continuing to manually depress the actuating lever of the tester in a slow, smooth and even manner until the test sample either fractured or began to plastically deform. The pressure, in psi, at which either of these events occurred was noted on the dial of the Gauge and recorded.

The following tables present the data that was obtained for mixtures of various sizes of granular bentonite.

Figure Legend:

BH=Big Horn Bentonite, Wyo-Ben, Inc's trade name for one of its Wyoming sodium bentonite products.

#8=particles in the range between 4 mesh (4,750 μm) and +12 mesh (1,700 μm) (−4+12 mesh)

#16=particles in the range between 8 mesh (2,360 μm) and 32 mesh (500 μm) (−8+32 mesh)

#30=particles between 12 mesh (1,700 μm) and 32 mesh (500 μm) (−12+32 mesh)

#40=particles less than 32 mesh (500 μm) (−32 mesh)

#34=a blend of approximately 55% #30 and 45% #40

#200=a fine ground product (powder) where approximately 80% of the particles are less than 200 mesh (75 μm) (80%-200 mesh)

Low adsorption bentonite=sodium bentonite that, because of its unique crystal structure and chemistry, has a significantly lower capacity to sorb water and swell, than other sodium bentonite and which is on the order of or slightly higher than the capacity of a typical calcium bentonite.

TABLE 3
SP Type 1/Test Type 1 - Pliability

| Sample | Pliability Index |
|---|---|
| BH #30 | 4 |
| BH #34 | 3.5 |
| BH #40 | 3 |
| BH #34 screened through 20 mesh sieve | 3.5 |
| (−20 mesh fraction of BH #34) | |
| 40% BH #30 + 60% BH #40 | 3.5 |
| #30 Low adsorption bentonite | 1.5 |
| #34 Low adsorption bentonite | 2 |
| #40 Low adsorption bentonite | 1 |
| 80% BH #34 + 20% #200 low adsorption bentonite | 3 |
| 80% BH #34 + 20% #200 calcium bentonite | 3.5 |

As shown in Table 3, this testing gauged the relative pliability of various compositions of granular sizes of bentonite product. Pliability/plasticity was deemed to be an asset for the inventive compositions described herein so that when the product is applied in the field, and if the wound is jostled or moved during transport (such as might occur under fire in battlefield situations), the wetted mass would not break in brittle fashion or pull free of the wound edges and allow re-bleeding but, rather, would move with the wound and stay firmly adhered to the wound. When compared with the observations from animal testing, using several of these same sample materials, Pliability Index values of about 3 to 3.5 were judged to be optimum.

TABLE 4
SP Type 2/Test Type 2 - Sorption

| | Time | Residual | | Average Break |
|---|---|---|---|---|
| | to sorb | Dry | Unsorbed | Pressure for 6 |
| | all water | Bentonite | Water | test specimens |
| Sample | (seconds) | (gm) | (cc) | (psi) |
| BH #30 | 10 | 11.37 | 0 | 3.6 |
| 95% BH #30 + 5% | 9 | 14.57 | 14.57 | — |
| BH #40 | | | | |
| 90% BH #30 + | 10 | 25.4 | 0 | — |
| 10% BH #40 | | | | |
| 85% BH #30 + | 25 | 30.9 | 0 | — |
| 15% BH #40 | | | | |
| 75% BH #30 + | 9 | 23.67 | 0 | 3.27 |
| 25% BH #40 | | | | |
| BH #16 | 9 | 0 | 0 | 3.68 |
| BH #8 | 9 | 0 | 0 | 3.58 |
| 80% BH #8 + 15% | 9 | 0 | 0 | 4.01 |
| BH #30 + 5% BH | | | | |
| #40 | | | | |
| Commercial Cat | 9 | 1.74 | 0 | 4.39 |
| Litter | | | | |
| 75% BH #30 + | 8 | 11.3 | 0 | 3.5 |
| 25% low | | | | |
| adsorption #40 | | | | |
| bentonite | | | | |
| #30 low adsorption | 7 | 0 | 0 | 1.25 |
| bentonite | | | | |

As shown in Table 4, this testing assessed various particle size compositions, as well as compositions of materials having different water adsorption characteristics, to identify a blend that had rapid water uptake, allowed wetting of most of the bentonite particles and generated moderate strength characteristics that would allow a mass of blood wetted product in a wound to retain its integrity and resist disintegration/fragmentation while still remaining flexible in the wound and adhered to the wound tissue. Break Pressure values of about 3.0 to 3.5 were judged to be optimum.

These tests, when viewed in the context of in vivo test results, demonstrated that the results obtained for BH #34 proved to be superior. However, as one of skill in the art will appreciate, other compositions possessing similar characteristics to those described herein are also encompassed within some embodiments of the invention described herein.

### EXAMPLE 2

An exemplary embodiment of the present invention was tested in vivo. It was the purpose of this study to test the performance of a proprietary mixture of the smectite mineral alone without the superabsorbant polymer in a lethal model of arterial hemorrhage against a predicate product. The predicate product chosen for comparison was QuikClot® granules.

Materials and Methods

This study was performed by North American Science Associates (NAMSA) of Northwood, Ohio. NAMSA is an AAALAC International accredited facility registered with the United States Department of Agriculture. It is also an FDA accredited Good Laboratory Practice facility. The study was approved by NAMSA's Institutional Animal Care and Use Committee and adhered to the National Institutes of Health Guide for the Care and Use of Laboratory Animals (National Institutes of Health publication 86-23, revised 1996).

Model and Animal Preparation:

The model described below is essentially identical to that developed and described by the Acheson et al. from the U.S. Army Institute of Surgical Research and duplicated by Ward and colleagues, in examining the earlier version of WS.^{8,9}

Fourteen Yorkshire crossbred commercial female swine (Sus scrofa domesticus) ranging in weight between 35-44 kg were utilized. Animals were fed a standard diet but fasted 12 hours prior to study with free access to water. On the day of study, animals were premedicated with a combination of tiletamine/zolazepam (4.4 mg/kg) and 2.2 mg/kg xylazine given intramuscularly before induction of anesthesia. Animals were intubated followed by maintenance of anesthesia with 2% isoflurane in oxygen and mechanically ventilated.

Mean arterial pressure (MAP) monitoring and blood sampling occurred via an arterial catheter surgically placed into the left carotid artery. Heart rate was monitored using a standard lead 3-electrocardiogram configuration. A 14-gauge catheter was placed in the jugular vein for fluid delivery. Upon obtaining vascular access, blood was sampled to perform baseline coagulation profiles (PT, aPTT, and complete blood count including a platelet count). Because blood loss in response to injury and treatment was an important outcome variable, animals underwent spleenectomy through a midline laparotomy in order to avoid the confounding variable of autotransfusion. After removal, the spleen was weighed and the animal was given three times the splenic weight in warmed lactated Ringers solution intravenously. The abdomen was then closed in an abbreviated fashion to minimize heat loss from the abdomen.

The left femoral artery was exposed via a large surgical incision made over the groin. The thin adductor muscle overlying the artery was removed using electrocautery. Approximately 5 cm of the artery was dissected free avoiding manipulation of the femoral nerve and vein. Small arterial branches emanating from the segment of the femoral artery were ligated. The artery was then clamped proximally and distally using vascular clamps. The entire length of the artery was then soaked in 2% lidocaine to further reduce chances of vasospasm. A 6 mm by 2 mm elliptical arteriotomy was created with an aortic vascular punch (Scanlan, Saint Paul, Minn.) leaving the posterior wall of the artery intact, which prevented retraction of the artery and vasospasm. The wound was expanded using a Weitlaner retractor to produce a large cavity in which blood could collect during hemorrhage. A temperature probe was secured at the base of the wound with suture in order to measure temperature changes produced during product application.

All animals were required to maintain a MAP greater than 60 mmHg after induction of anesthesia to be included in the study. Bleeding was induced by release of the vascular clamps. Free bleeding took place for 45 seconds. Blood spilling out of the cavity was suctioned into pre-weighed canisters. Pre-weighed absorbent pads placed under the animal also collected blood that was not suctioned. Blood collected prior to product application was measured and counted as pre-treatment blood loss (PreTBL).

After 45 seconds of free bleeding, animals were randomized to be treated with either 3.5 ounces Quick Clot® granules (QCG) (obtained from North American Rescue Products, Inc. Greenville, S.C.), or 5.5 ounces of an embodiment of the present invention (a version of the WoundStat™ product, is available from TraumaCure, Bethesda, Md., herein referred to as WS). Both products are granular and were placed through the accumulated pool of blood in the wound. The application of QCG followed the manufacturer's directions, which included pouring the product into the wound followed by application of direct pressure. Application of WS followed the manufacturer's directions, which included packing of WS into all areas of the wound followed by application of direct pressure. Total application and pressure time was 3 minutes after which time pressure was discontinued. If bleeding was observed, the product was removed from the wound and a fresh application of the same product was placed in the wound in an identical fashion as described above. After this time, pressure was discontinued and the wound was left undisturbed. Animals were monitored for 2 hours or until death. During this time, if animals began to hemorrhage from the wound site around the product, blood was collected either by suction or from newly placed pre-weighed absorbent pads that had been placed at the time of the first application of the product. All blood collected after the first application of the product was counted as post-treatment blood loss (Post-T2L).

At the time of the first application of the product, animals were given a 500 cc bolus of Extend® solution (6% Heptastich in a balanced salt solution) (Abbott Laboratories, Abbott Park, Ill.) followed by administration of pre-warmed lactated Ringers solution at a rate of 100 mol/min whenever the mean arterial blood pressure (MAP) dropped below 65 mmHg. A target MAP of 65 mmHg was chosen as it has been previously demonstrated to be above a threshold pressure that promotes rebleeding.¹⁰ The total amount of fluid provided for each animal during and after injury was recorded.

Animals were observed for 2 hours after product application. Animals surviving to 2 hours were euthanized using intravenous sodium pentobarbital.

Statistical Analysis:

Data are expressed as means±SD. Statistical significance was set at a p value of <0.05. Pre-injury parameters (MAP, weight, hematocrit, coagulation parameters) between groups were compared using unpaired t tests. Comparisons of pre- and post-treatment blood loss, resuscitation fluid volumes, and temperature were performed using the Mann Whitney test (non-parametric t test). Fisher's exact test was used to determine significant differences occurring in the incidence of initial hemostasis and survival. Survival times were analyzed using the Logrank test. Data analysis was performed using the statistical software package GraphPad Instat and GraphPad Prism (Graphpad, San Diego, Calif.)

Results

Table 5 lists baseline data of all groups. All animals qualified for the study and no significant difference was found to exist in baseline parameters among groups.

TABLE 5
Baseline weight, hemodynamic, and coagulation parameters of groups.

| | | | Baseline | | | |
|---|---|---|---|---|---|---|
| | | Hematocrit | MAP | Platelet count | | aPTT |
| | Wt (kg) | (%) | (mmHg) | (10⁹/L) | PT (sec) | (sec) |
| QCG | 38.6 ± 2.8 | 30.6 ± 2.7 | 71.7 ± 15.4 | 188.3 ± 61.6 | 12.9 ± 7.1 | 15.7 ± 2.2 |
| WS | 40 ± 2.7 | 30.5 ± 1.9 | 72.7 ± 4.2 | 229.2 ± 70.2 | 10.3 ± 0.6 | 16.4 ± 4 |
| P | 0.34 | 0.88 | 0.87 | 0.30 | 0.34 | 0.68 |
| Value | (NS) | (NS) | (NS) | (NS) | (NS) | (NS) |

NS = Not statistically significant
Table Legend: (WS) WoundStat ™, (QCG) QuikClot ® Granules, (Wt) Weight, (MAP) Mean Arterial Pressure, (PT) Prothrombin Time, (aPTT) Activated Partial Thromboplastin Time

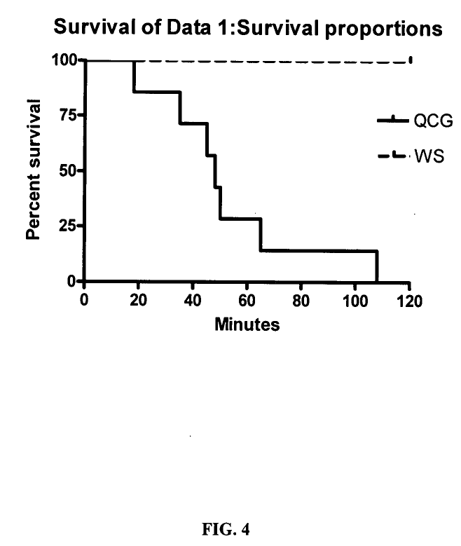

Table 6 provides a comparison of pertinent parameters among groups after the start of hemorrhage and the post product application time period. There was no significant difference among groups in Pre-TBL or pre-application MAP. All animals receiving WS achieved complete hemostasis. A second application of product was not required for any animal in the WS group. All animals receiving QCG demonstrated profound bleeding after the first application of product, necessitating a second application. Despite a second application, hemostasis could not be achieved. There was a 100% survival rate in the WS group to 180 minutes compared to no survivors in the QCG group (p=0.0005). Survival time for the WS group was significantly higher compared to the QCG group (p=0.001) (FIG. 4).

TABLE 6
Post-injury hemostatic, hemodynamic, resuscitation, and survival differences between groups

| | | | | Pre- | | | | Survival |
|---|---|---|---|---|---|---|---|---|
| | | | PreMAP | TBL | Post-TBL | Post LR | Survival | time |
| | Hemostasis | | (mmHg) | (ml/kg) | (ml/kg) | (ml/kg) | (%) | (min) |
| QCG | 0/7 | (0%) | 50.7 ± 10.4 | 12.5 ± 7 | 121.5 ± 34.3 | 156.6 ± 100.3 | 0% | 52.7 ± 28.3 |
| WS | 7/7 | (100%) | 44.3 ± 15.2 | 11.2 ± 5.4 | 0.0 ± 0.0 | 39.6 ± 23.4 | 100% | 120 ± 0.0 |
| P | 0.0005 | (S) | 0.37 | 0.73 | 0.0043 | 0.0041 | 0.0005 | 0.0001 |
| Value | | | (NS) | (NS) | (S) | (S) | (S) | (S) |

Table Legend: Pre Mean Arterial Pressure (MAP) is MAP at end of 45 second hemorrhage but before product application. Pretreatment blood loss (Pre-TBL) is total blood loss just before application of product. Post-treatment blood loss (Post-TBL) is total blood loss after application of product. Post-LR is the volume of lactated Ringers given post-application to maintain MAP of 65 mmHg. (WS) WoundStat ™, (QCG) QuikClot ® Granules

As noted in Table 6, there was a significant decrease in total Post-TBL in the WS group compared to the QCG group. Concordantly, the amount of post-application lactated Ringers required to maintain a target MAP of 65 mmHg was significantly less in the WS group compared to QCG group.

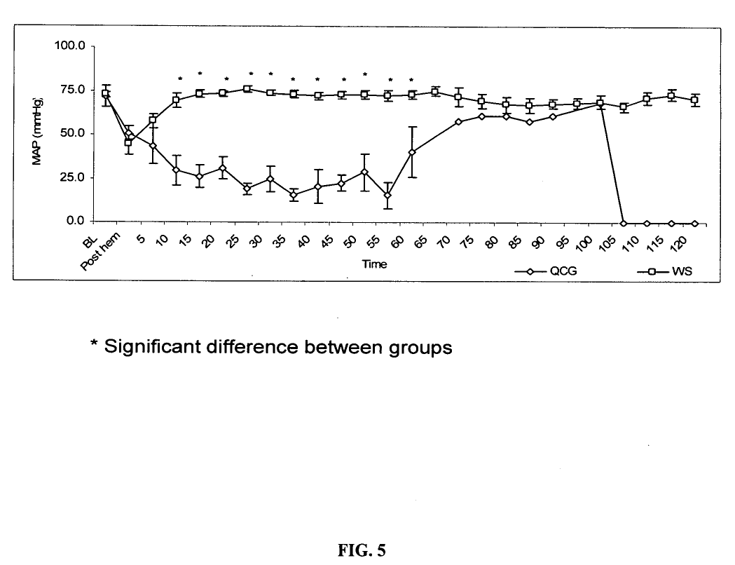

FIG. 5 depicts the average MAP over time for the two groups. Significant differences between WS and QCG were noted as early as 15 minutes post-application. This difference became transiently insignificant at approximately 70 minutes when the one QCG animal surviving to that point was able to increase its blood pressure temporarily before experiencing cardiovascular collapse.

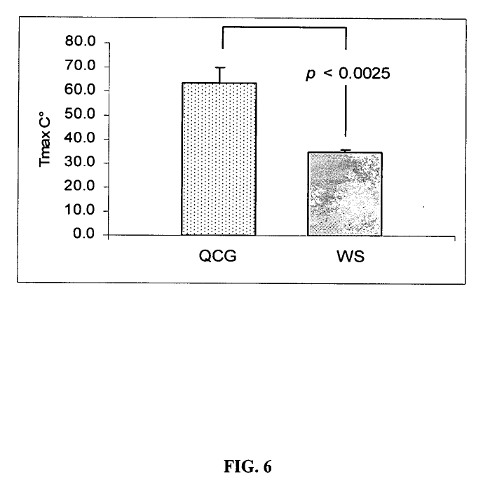

FIG. 6 demonstrates the difference in peak wound temperature between the two groups. QCG produced peak temperatures of 63.6±17.4° C. compared to 33.4±4.7° C. produced by WS (p<0.0025).

Discussion

The current study demonstrated that the WS product consisting the smectite mineral alone (without the polyacrylate) yielded identical survival and similar post-application blood loss results to the earlier combination product and was significantly better than QCG in producing hemostasis, survival to two hours, and in reducing fluid resuscitation to maintain an MAP of 65 mmHg.⁸ These results are also similar to the studies by Acheson et al. and Ward et al., who found no survival benefit of QCG in the model of lethal arterial hemorrhage described in this study.^{8,9}

The model used in this study first reported by Acheson et al. and then by Ward et al. represents what may be considered an extreme challenge for a hemostatic agent.^{8, 9} In this model, the injury to the artery does not allow the artery to retract or vasospasm, and thus achieve hemostasis spontaneously. Coupled with immediate volume resuscitation, which rapidly restores MAP close to normal values, any hemostatic agent faces a significant hydrostatic challenge in its ability to induce a stable clot or seal in a short period of application time. Placement of agents directly through a pool of blood probably adds an additional challenge.⁷

Although this model may represent an extreme and may lack other relevant components of a combat acquired wound, such as venous bleeding and surrounding soft tissue injury, the model is highly reproducible and may represent a worst case scenario. Alam et al. have produced a different complex groin injury in which both the femoral artery and vein are completely transected. Hemorrhage is allowed to occur for 3-5 minutes, which reduces the MAP to a greater degree than in the Acheson model.^{11, 12} Product application follows with pressure held for five minutes and fluid resuscitation begun 15-30 minutes post-injury. In this model, the major source of bleeding at the time of product application is considered to be venous in nature because the artery has spasmed and retracted. The model is still 100% lethal if not treated, but has a greater than 60% survival rate when treated with only standard gauze. Alam and colleagues have demonstrated a significant improvement in survival using QCG over no dressing using this model, but have not been able to distinguish statistically significant survival benefits of QCG over standard gauze dressing.^{11, 12}

The depth and irregular geometry of combat wounds make uniform application and acceptable performance of a hemostatic agent difficult even under the best of conditions, but especially when applied by non-medical personnel. When additional circumstances are added, such as wounds occurring in places that are not amenable to tourniquet application and the inability to hold pressure for extended periods of time, the challenge of a hemostatic agent to perform is daunting. It is with these issues in mind—along with the criteria outlined by Pusateri et al.—that we developed the current WS product.⁷ Because of the great potential for deep wounds and irregular wound geometry, we wanted to create a safe and effective product that would address a number of unique challenges on the battlefield and in major civilian traumas. First, we focused on a granular hemostatic agent heavy enough to be poured into the wound without being rapidly flushed away by ongoing bleeding or easily blown away in adverse weather conditions. Second, we wanted to ensure product contact with the site(s) of bleeding and conformance to the wound. Third, recognizing the potential limited access to additional product in emergency evacuation situations, we needed to assure that the product could be re-applied if bleeding recurred.

The previous version of WS included a smectite mineral, which is a from a class of hydrated alumino silicates with excellent absorption and packing properties, and a salt of a crosslinked polyacrylic acid, which is capable of rapidly absorbing over 200 times its weight in water.¹³⁻¹⁵ The combined properties of the smectite mineral and the polymer of the previous WS product resulted in extremely fast absorption of blood as well as significant tissue adherence. However, upon further study to investigate its robustness and flexibility in situations that might be envisioned in combat, we found that the initial formulation of WS could not be reused to stop bleeding. It appeared that the formulation was initially spent upon first application and that if rebleeding occurred, the material in the wound could not absorb the additional blood. To stop the bleeding, the combination product needed to be removed and a fresh application needed to be applied. Furthermore, we found that adding additional product on top of the initial packing was not as effective as the new material and could not be mixed with the already spent material in the wound. Results in our laboratories (data not reported here) demonstrated that using just the smectite mineral component overcame these issues making the product potentially more robust and flexible. Thus, we conducted the current study to ensure that the smectite only product would perform at least as well as the initial formulation.

Several of the WS product's properties indicate that the product has a significant negative electrostatic charge, which may assist in activating the intrinsic clotting system.^{13, 16} This mechanism differs from the cationic charge reported for chitosan, which is believed to result in red cell aggregation and clot promotion.^{17, 18} Additionally, the rapid absorption of blood by the WS mixture may help in concentrating red cells and clotting factors at the site of injury. Given the rapid ability to achieve hemostasis, WS is likely most effective through its ability to be packed into the wound rapidly and firmly, to form a seal over the bleeding sites, and conform to all surfaces of the wound cavity. The mechanism of rapid absorption and concentration of clotting factors has also been suggested by the manufacturer of QuikClot® as the major mechanism of action for the QuikClot® products. However, the QCG product results in a significant exothermia capable of producing tissue injury consistent with severe burns.^{9, 19-21} Attempts to reduce the exothermia of QuikClot by adding residual moisture have failed to improve its efficacy in less severe models.¹¹

In summary, WS consisting only of the smectite mineral was superior in achieving hemostasis, prolonging survival to two hours, and reducing post-hemorrhage fluid requirements in a lethal model of arterial hemorrhage compared to QCG. The WS product would appear to meet many of the criteria set forth by Pusateri et al. as an ideal hemostatic agent.⁷

### REFERENCES

1. Bellamy R F. The causes of death in conventional land warfare: implications for combat casualty care research. Mil Med 1984; 149:55-62.
2. Champion H R, Bellamy R F, Roberts C P, Leppaniemi A. A profile of combat injury. J Trauma 2003; 54:S13-9.
3. Sauaia A, Moore F A, Moore E E, et al. Epidemiology of trauma deaths: a reassessment. J Trauma 1995; 38:185-93.
4. Sauaia A, Moore F A, Moore E E, Haenel J B, Read R A, Lezotte D C. Early predictors of postinjury multiple organ failure. Arch Surg 1994; 129:39-45.
5. Cosgriff N, Moore E E, Sauaia A, Kenny-Moynihan M, Burch J M, Galloway B. Predicting life-threatening coagulopathy in the massively transfused trauma patient: hypothermia and acidoses revisited. J Trauma 1997; 42:857-61; discussion 861-2.
6. Heckbert S R, Vedder N B, Hoffman W, et al. Outcome after hemorrhagic shock in trauma patients. J Trauma 1998; 45:545-9.
7. Pusateri A E, Holcomb J B, Kheirabadi B S, Alam H B, Wade C E, Ryan K L. Making sense of the preclinical literature on advanced hemostatic products. J Trauma 2006; 60:674-82.
8. Ward K R, Tiba M H, Holbert W R, et al. Comparison of a new hemostatic agent to current combat hemostatic agents in a Swine model of lethal extremity arterial hemorrhage. J Trauma 2007; 63:276-83; discussion 283-4.
9. Acheson E M, Kheirabadi B S, Deguzman R, Dick E J, Jr., Holcomb J B. Comparison of hemorrhage control agents applied to lethal extremity arterial hemorrhages in swine. J Trauma 2005; 59:865-74; discussion 874-5.
10. Sondeen J L, Coppes V G, Holcomb J B. Blood pressure at which rebleeding occurs after resuscitation in swine with aortic injury. J Trauma 2003; 54:S110-7.
11. Alam H B, Chen Z, Jaskille A, et al. Application of a zeolite hemostatic agent achieves 100% survival in a lethal model of complex groin injury in Swine. J Trauma 2004; 56:974-83.
12. Alam H B, Uy G B, Miller D, et al. Comparative analysis of hemostatic agents in a swine model of lethal groin injury. J Trauma 2003; 54:1077-82.
13. Odom I E. Smectite clay minerals: Properties and uses. Philosophical Transactions of the Royal Society of London. Series A, Mathematical and Physical Sciences 1984; 311:391-409.
14. Omidian H, Rocca J G, Park K. Advances in superporous hydrogels. J Control Release 2005; 102:3-12.
15. Askari F, Nafisi S, Omidian H, Hashemi S. Synthesis and characterization of acrylic-based superabsorbents. Journal of Applied Polymer Science 1993; 50:1851-1855.
16. Moskowitz K. Procoagulant assessment of topical battlefield hemotstats using a novel semi-automated whole blood clotting assay system (abstract). Blood 2007; 110:3161.
17. Thatte H S, Zagarins S E, Amiji M, Khuri S F. Poly-N-acetyl glucosamine-mediated red blood cell interactions. J Trauma 2004; 57:S7-12.
18. Suh J K, Matthew H W. Application of chitosan-based polysaccharide biomaterials in cartilage tissue engineering: a review. Biomaterials 2000; 21:2589-98.
19. Alam H B, Burris D, DaCorta J A, Rhee P. Hemorrhage control in the battlefield: role of new hemostatic agents. Mil Med 2005; 170:63-9.
20. Pusateri A E, Delgado A V, Dick E J, Jr., Martinez R S, Holcomb J B, Ryan K L. Application of a granular mineral-based hemostatic agent (QuikClot) to reduce blood loss after grade V liver injury in swine. J Trauma 2004; 57:555-62; discussion 562.
21. Wright J K, Kalns J, Wolf E A, et al. Thermal injury resulting from application of a granular mineral hemostatic agent. J Trauma 2004; 57:224-30.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A unit package comprising a measured amount of a sterile composition comprising a mixture of clay particles, wherein the particles are of at least two different specified particle sizes and the mixture provides at least one of the following activities: sorbing a body fluid, forming a cohesive mass, adhering to tissue, sealing a wound, promoting coagulant activity, and stopping blood flow from a wound.

2. The unit package of claim 1, wherein the clay is selected from a group consisting of bentonite, montmorillonite, beidelite, nontronite, saponite, hectorite, illite, illite-smectite mixed layer clay, sepiolite, attapulgite (palygorskite), kaolin, kaolinite, and mixtures thereof.

3. The unit package of claim 1, wherein the fluid sorbed is blood or a wound fluid.

4. The unit package of claim 1, wherein the mixture of clay particles forms an adherent seal by mixing with blood to create a pliable cast within a wound that applies pressure to the wound.

5. The unit package of claim 1, wherein the unit package is suitable for medical use and comprises a measured amount of a sterile composition comprising a loose mixture of clay particles, wherein at least about 90% of the particles have a particle size of less than 12 mesh.

6. The unit package of claim 1, wherein the unit package is suitable for medical use and comprises a measured amount of a sterile composition comprising a mixture of loose clay particles, wherein at least about 95% of the particles have a particle size of less than 12 mesh and about 10% of the particles have a particle size of less than 100 mesh.

7. The unit package of claim 1, wherein the unit package is suitable for medical use and comprises a measured amount of a sterile composition comprising a mixture of loose clay particles, wherein at least about 100% of the particles have a particle size of less than 12 mesh, about 35% to about 50% of the particles have a particle size of less than 40 mesh, and about 15% of the particles have a particle size of less than 100 mesh.

8. The unit package of claim 1, wherein the package contains multiple units, each unit containing s sufficient amount of the clay mixture for application to a wound.

9. The unit package of claim 1, wherein the measured amount is between 2 ounces and 10 ounces.

10. The unit package of claim 1, wherein the measured amount is between 2 ounces and 6 ounces.

11. The unit package of claim 1, wherein the measured amount is between 0.01 gram to about 100 grams.

12. The unit package of claim 1, wherein the measured amount is between 1 gram to about 50 grams.

13. The unit package of claim 1, wherein the measured amount is between 50 grams and 250 grams.

14. The unit package of claim 1, wherein the unit package is sterilized.

15. The unit package of claim 1, wherein the composition has a specified moisture content.

16. The unit package of claim 15, wherein the moisture content is between about 5% to about 13%.

17. A method of producing a unit package comprising a measured amount of a sterile composition comprising a mixture of small clay particles and large clay particles comprising, without regard to order:
(a) selecting a measured amount of clay particles that will pass through a 4 mesh sieve;
(b) selecting a measured amount of clay particles from (a) that will be retained on a 100 mesh sieve; and
(c) sterilizing the measured amount of the mixture to form a measured amount of a sterile composition and packaging the sterile composition into a unit package; or
(d) packaging the measured amount of the mixture into a unit package and sterilizing the unit package and mixture.

18. A method of promoting hemostasis in a hemorrhaging wound, comprising applying the sterile composition of claim 1 in a quantity sufficient to promote one or both of the following: i) hemostasis and ii) formation of a cast comprising the one or more clay minerals and blood from said hemorrhaging wound.

19. The method of claim 18, wherein the cast forms a seal over a point of rupture in a blood vessel.

20. The method of claim 18, wherein the cast forms a seal over the wound.

21. The method of claim 18, wherein the cast forms a seal over the wound and also over a point of rupture in a blood vessel.

22. The method of claim 18, wherein the composition is applied directly to the wound.

23. The method of claim 22, wherein the composition is loose particles of clay.

24. The method of claim 18, wherein the composition is attached to or contained within a substrate.

25. The method of claim 24, wherein the substrate is selected from the group consisting of cotton, wool, linen, rayon, nylon, polyester, polyethylene, mineral wool or metal fibers, a dissolvable material, a water soluble material, and blends of these materials.

26. The method of claim 18, wherein the wound is either internal or external.

27. The method of claim 18, wherein the cast consists essentially of wound fluids and blood mixed with clay.

28. The method of claim 18, wherein the cast is pliable and durable, and capable of remaining structurally intact.

29. The method of claim 18, wherein the cast forms an adhesive seal within the wound.

30. A method of forming a cast to cover a hemorrhaging wound, comprising applying the sterile composition of claim 1 in a quantity sufficient to form a cast comprising one or more clay minerals and blood from said hemorrhaging wound.

31. The method of claim 30, wherein the composition is applied directly to the wound.

32. The method of claim 30, wherein the cast is administered to a coagulopathic mammal.

33. The method of claim 30, wherein the cast consists essentially of wound fluids and blood mixed with clay.

34. The method of claim 30, wherein the cast is pliable and durable, and capable of remaining structurally intact.

35. The method of claim 30, wherein the cast forms an adhesive seal within the wound.

36. A method of selecting a hemostatic composition comprising selecting a particle size mixture; testing the particle size mixture for at least one of the following: cohesion between particles, absorption, adsorption, or pliability; analyzing the test results to select the components of a mixture; and producing a hemostatic composition comprising the mixture.

37. A method of forming a cast comprising applying directly to a wound a measured amount of a sterile composition comprising a mixture of clay particles, wherein the particles are of different specified particle sizes, to form a cast that adheres to the wound.

38. The method of claim 37, wherein the cast consists essentially of wound fluids and blood mixed with particles of clay.

39. A unit package comprising a measured amount of a sterile composition comprising a mixture of clay particles, wherein the particles are of at least two different specified particle sizes and the mixture forms a cohesive, pliable mass after sorbing a fluid.

40. The unit package of claim 39, wherein the fluid is blood.

41. The unit package of claim 39, wherein the wetted mass stops the flow of blood from a wound in a mammal.

42. The unit package of claim 41, wherein the mammal is a human.

43. The unit package of claim 41, wherein the cohesive, pliable mass is durable and remains structurally intact when moving the mammal.

44. The unit package of claim 39, wherein the clay mixture is loose particles of clay.

45. The unit package of claim 39, wherein the clay mixture is fixed to a substrate.

46. The unit package of claim 39, wherein the substrate is selected from the group consisting of cotton, wool, linen, rayon, nylon, polyester, polyethylene, mineral wool or metal fibers, a dissolvable material, a water soluble material, and blends of these materials.

47. The unit package of claim 39, wherein the packaged mixture is enmeshed in the substrate, intertwined with the substrate, coated onto the substrate, contained within, or adhered to the substrate.

48. The unit package of claim 45, wherein the substrate comprises polyvinyl alcohol, ethylcellulose, hydroxypropyl methylcellulose, polyethylene oxide, or mixtures thereof.

49. The unit package of claim 46, wherein the water soluble material is a water soluble plastic.
